(19) United States
(12) Patent Application Publication
(10) Pub. No.: US 2016/0317113 A1
(43) Pub. Date: Nov. 3, 2016

(54) DETERMINING THE VELOCITY OF A FLUID USING AN IMAGING METHOD

(71) Applicant: Siemens Aktiengesellschaft, Muenchen (DE)

(72) Inventors: Thomas ALLMENDINGER, Forchheim (DE); Thomas FLOHR, Uehlfeld (DE); Gregor JOST, Berlin (DE); Hubertus PIETSCH, Kleinmachnow (DE); Bernhard SCHMIDT, Fuerth (DE)

(73) Assignee: Bayer Pharma Aktiengesellschaft, Berlin (DE)

(21) Appl. No.: 15/132,365

(22) Filed: Apr. 19, 2016

(30) Foreign Application Priority Data
Apr. 29, 2015 (DE) 102015207894.9

(51) Int. Cl. A61B 6/00; A61B 6/03

(52) U.S. Cl. CPC A61B 6/507; A61B 6/032; A61B 6/481

(57) ABSTRACT

A method is described for determining the velocity of a fluid in a region to be investigated using an imaging method, preferably computer tomography, of an investigation object. In the method, a plurality of separately spaced sub regions of a region to be investigated, through which sub regions the fluid is flowing, are defined. Time-dependent image data is produced for the plurality of separately spaced sub regions. Moreover, time/density curves are produced with, in each case, a plurality of time-dependent intensity values on the basis of the time-dependent image data for the separately spaced sub regions. Additionally, the time displacement in the time/density curves is determined. Lastly, the fluid velocity is determined on the basis of the time displacement determined in the time/density curves. A fluid velocity determination device is also described. Moreover, a computer tomography system is described.

### PRIORITY STATEMENT

The present application hereby claims priority under 35 U.S.C. §119 to German patent application number DE 102015207894.9 filed Apr. 29, 2015, the entire contents of which are hereby incorporated herein by reference.

### FIELD

At least one embodiment of the invention generally relates to a method for determining the velocity of a fluid in a volume to be mapped using an imaging method, preferably computer tomography, of an investigation object. Additionally, at least one embodiment of the invention generally relates to a fluid velocity determination device. Furthermore, at least one embodiment of the invention generally relates to a computer tomography system.

### BACKGROUND

Modern imaging methods are frequently employed to generate two-dimensional or three-dimensional image data which can be used for visualizing a mapped investigation object, and moreover also for other applications.

The imaging methods are frequently based on capturing X-ray radiation, wherein so-called projection measurement data is generated. For example, projection measurement data can be acquired by using a computer tomography system (CT system). In CT systems, a combination of an X-ray source and an oppositely arranged X-ray detector, the combination being arranged on a gantry, usually rotates around a measurement space in which the investigation object (which is referred to in the following, without restricting its generality, as the patient) is situated. In this regard, the center of rotation (also called the “isocenter”) coincides with a so-called system axis, also called the z-axis, which extends in the z-direction. During one or a plurality of rotations, the patient is exposed to X-ray radiation from the X-ray source, projection measurement data or X-ray projection data being captured by using the oppositely located X-ray detector.

The projection measurement data generated is especially dependent on the design of the X-ray detector. X-ray detectors usually have a plurality of detection units which are arranged for the most part in the form of a regular pixel array. The detection units generate a detection signal in each case for any X-ray radiation striking the detection units, which signal is analyzed at certain time points in terms of intensity and spectral distribution of the X-ray radiation in order to obtain conclusions about the investigation object and generate projection measurement data.

For a long time, it was the case that “only” anatomical structures could be reproduced in image form by using CT imaging. On the other hand, functional imaging by way of computer tomography was impossible for a long time, among other things due partly to an excessively high dose uptake for the patient. Owing to technological advances, however, the opportunities for functional imaging have improved and found their way into the clinical routine in the last few years.

Modern CT systems permit the recording of four-dimensional image data for functional imaging. Depending on the recording technology, the dimensions of regions to be mapped in the z-direction, i.e. in the direction of the system axis, which also coincides with the longitudinal axis of the patient, can correspond to the width of the detector used in the case of a fixed table position, or be dimensioned substantially larger in the case of a periodically moving patient table. There are various ways of analyzing the image data captured in this way. For example, the image data produced can be visualized as four-dimensional image data. In this regard, the time point and the level of blood flow through vessels can be represented in color. It is therefore possible to represent graphically, in a three-dimensional image for example, if vessel regions are supplied with blood substantially later. Moreover, a functional analysis of the parenchyma, i.e. of the functional tissue, can also be carried out.

In the case of functional imaging, there is also interest in the determination of fluid velocities and especially also of blood flow velocity.

On the one hand, knowledge about blood flow velocities can help in finding and/or characterizing pathologies (e.g. stenoses). On the other hand, it enables the optimization of acquisition parameters in the case of CT scans supported by contrast media, such as angiographies for example.

Identification of blood flow velocity is already possible with medical measurement methods such as magnetic resonance tomography (MRT) and ultrasound (US) for example. In cases where blood flow velocity is identified by using magnetic resonance tomography, the body tissue is put into a specific electromagnetic state by way of magnetic fields. The velocity of the blood is then identified from the change in magnetization, e.g. due to the blood flow (“magnetic resonance velocimetry”). Contrast media are not always necessary for these methods.

In cases where the blood flow velocity is identified by using an ultrasound method, on the other hand, use is made of the Doppler effect, wherein the frequency shift in the sound waves expresses the level of the blood flow velocity. No contrast media are necessary in the case of this method either, and in a similar manner there are also optical methods (e.g. with lasers) to measure the blood flow velocity via the Doppler effect.

On the other hand, determination of blood flow velocity and other fluid velocities in the case of CT imaging has only been achievable to a limited extent up to now due to technical constraints.

In the case of CT imaging, temporal resolution is very limited and is additionally dependent on the rotational velocity of the gantry. This makes it more difficult to determine the blood flow velocity especially if the coverage, i.e. the detector dimensions in the z-direction, i.e. in the direction of the system axis, is small. In other words, the accuracy of fluid velocity measurement is dependent on how the detector is dimensioned in the z-direction: the smaller the detector, the worse the accuracy. Additionally, in the case of measurements of blood flow velocity based on only a few measured values as a function of time, artifacts and a fairly unfavorable signal/noise ratio make it harder to determine the blood flow velocity on the basis of those measured values. Moreover, non-equidistant scanning and scanning as a function of the z-position make it harder to determine fluid velocity since it is necessary to analyze data points that are not synchronized to each other.

### SUMMARY

At least one embodiment of the present invention is directed to a method for determining a fluid velocity in a region of the body to be investigated, which method can also be applied with sufficient accuracy with the aid of conventional CT machines.

At least one embodiment is directed to a method for determining the velocity of a fluid, a fluid velocity determination device; and/or a computer tomography system.

In at least one embodiment of the inventive method for determining the velocity of a fluid in a volume to be mapped using an imaging method, preferably computer tomography, of an investigation object, a plurality of separately spaced sub regions of the region to be investigated, through which sub regions the fluid is flowing, are defined. In order to define the sub regions, a setting of the imaging system employed for the imaging method is usually undertaken in advance, for example on the basis of information determined in advance about the position of the sub regions to be recorded. For this purpose, an overview image can be recorded in advance for example, in which the bodily structures of a patient can be broadly recognized. Following definition of the separately spaced sub regions to be recorded, time-dependent image data is recorded for the plurality of separately spaced sub regions with the imaging method. On the basis of the time-dependent image data, time/density curves are determined with, in each case, a plurality of time-dependent intensity values for the separately spaced sub regions. In other words, a time/density curve represents, in each case, the time-dependent intensity values captured during the imaging method for one assigned sub region in each case. During the determination of the time/density curves, the intensity values assigned to a respective sub region can be averaged over the surface of the respective sub region and the time/density curve can be determined on the basis of these averaged intensity values.

At least one embodiment of an inventive fluid velocity determination device comprises a region definition unit for defining a plurality of separately spaced sub regions of a region to be investigated, through which sub regions the fluid is flowing. At least one embodiment of the inventive fluid velocity determination device also comprises an image data capture unit for producing time-dependent image data for the plurality of separately spaced sub regions. An image data capture unit of this type usually has functions for capturing raw data or projection measurement data and reconstructing image data on the basis of the captured raw data. The inventive fluid velocity determination device also comprises a curve determination unit for determining time/density curves with, in each case, a plurality of time-dependent intensity values on the basis of the time-dependent image data for the separately spaced sub regions. Forming part of the inventive fluid velocity determination device are also a displacement determination unit for determining the time displacement in the time/density curves and a velocity determination unit for determining the fluid velocity on the basis of the time displacement determined in the time/density curves.

At least one embodiment of an inventive computer tomography system encompasses the inventive fluid velocity determination device.

At least one embodiment of the inventive computer tomography system additionally encompasses, for example, a projection data acquisition unit. The projection data acquisition unit comprises an X-ray source and a detector system for acquiring projection measurement data from an object. Furthermore, at least one embodiment of the inventive computer tomography system also comprises a reconstruction unit for reconstructing captured projection measurement data and additionally the inventive fluid velocity determination device, wherein, in the case of at least one embodiment of the inventive computer tomography system, the reconstruction unit preferably forms part of the fluid velocity determination device.

The fundamental components of at least one embodiment of the inventive fluid velocity determination device can be realized in the majority of cases in the form of software components. This relates especially to the region definition unit, parts of the image data capture unit, the curve determination unit, the displacement determination unit, and the velocity determination unit. In principle, however, these components can also be implemented partly in the form of software-supported hardware, for example FPGAs or the like, especially if particularly fast calculations are involved. Likewise the required interfaces can be realized as software interfaces, for example if only the importing of data from other software components is involved. But they can also be realized as interfaces constructed using hardware, which are activated via suitable software.

At least one embodiment of the inventive fluid velocity determination device can especially form part of a user terminal or a control device of a CT system.

A largely software-based implementation has the advantage that previously used control devices can also be retrofitted in a simple manner via a software update in order to operate in the inventive manner. To this extent, at least one embodiment is directed to a corresponding computer program product with a computer program, which is capable of being loaded directly into a memory device of a control device of a computer tomography system, containing program sections in order to execute all the steps of at least one embodiment of the inventive method, when the program is executed in the control device. Where appropriate, a computer program product of this type can comprise, apart from the computer program, additional elements such as documentation for example and/or additional components also hardware components such as hardware keys (dongles etc.) for example, for the purpose of using the software.

For transport to the control device and/or for storage on or in the control device, use can be made of a computer-readable medium, for example a memory stick, a hard drive or some other transportable or permanently installed data medium, on which the program sections of the computer program that are capable of being read and executed by an arithmetic and logic unit of the control unit are stored. The arithmetic and logic unit can encompass one or a plurality of interoperating microprocessors or the like for this purpose, for example.

The dependent claims and also the following description respectively contain especially advantageous embodiments and developments of the invention. In this regard, especially, the claims in one claim category can also be developed analogously to the dependent claims in another claim category.

Additionally, the various features of different example embodiments and claims can also be combined into new example embodiments in the context of the invention.

### BRIEF DESCRIPTION OF THE DRAWINGS

In the following, the invention is explained in detail again on the basis of example embodiments by referring to the enclosed figures. These comprise the following:

FIG. 1 a flow diagram which illustrates a method for determining a fluid velocity according to an example embodiment of the invention,

FIG. 2 the definition of a plurality of sub regions to be mapped,

FIG. 3 the time profile of a plurality of contrast medium curves,

FIG. 4 a perspective view of a leg containing an artery which is oriented in the z-direction along the z-axis of a CT system, and a plurality of sub regions to be mapped which are situated at various positions on the z-axis,

FIG. 5 a graph containing a plurality of time/density curves which are assigned to the sub regions to be mapped as shown in FIG. 4,

FIG. 6 a graph which illustrates the distribution of the maxima in the time/density curves represented in FIG. 5 in the location/time plane and also the determination of a central time displacement in the time/density curves,

FIG. 7 a block diagram representing a fluid velocity determination device according to an example embodiment of the invention,

FIG. 8 a schematic representation of computer tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In at least one embodiment of the inventive method for determining the velocity of a fluid in a volume to be mapped using an imaging method, preferably computer tomography, of an investigation object, a plurality of separately spaced sub regions of the region to be investigated, through which sub regions the fluid is flowing, are defined. In order to define the sub regions, a setting of the imaging system employed for the imaging method is usually undertaken in advance, for example on the basis of information determined in advance about the position of the sub regions to be recorded. For this purpose, an overview image can be recorded in advance for example, in which the bodily structures of a patient can be broadly recognized. Following definition of the separately spaced sub regions to be recorded, time-dependent image data is recorded for the plurality of separately spaced sub regions with the imaging method. On the basis of the time-dependent image data, time/density curves are determined with, in each case, a plurality of time-dependent intensity values for the separately spaced sub regions. In other words, a time/density curve represents, in each case, the time-dependent intensity values captured during the imaging method for one assigned sub region in each case. During the determination of the time/density curves, the intensity values assigned to a respective sub region can be averaged over the surface of the respective sub region and the time/density curve can be determined on the basis of these averaged intensity values.

Furthermore, a time displacement is determined in the time/density curves, which are assigned to different sub regions, relative to each other. Since the different sub regions are arranged at different positions, time-displaced profiles are also created for the assigned time/density curves. In more accurate terms, the time displacement is dependent on the spacing between the sub regions and the fluid velocity. Conversely, the fluid velocity can be calculated on the basis of the time displacement determined in the time/density curves and also the known spacing between the sub regions to which the individual time/density curves are assigned.

At least one embodiment of an inventive fluid velocity determination device comprises a region definition unit for defining a plurality of separately spaced sub regions of a region to be investigated, through which sub regions the fluid is flowing. At least one embodiment of the inventive fluid velocity determination device also comprises an image data capture unit for producing time-dependent image data for the plurality of separately spaced sub regions. An image data capture unit of this type usually has functions for capturing raw data or projection measurement data and reconstructing image data on the basis of the captured raw data. The inventive fluid velocity determination device also comprises a curve determination unit for determining time/density curves with, in each case, a plurality of time-dependent intensity values on the basis of the time-dependent image data for the separately spaced sub regions. Forming part of the inventive fluid velocity determination device are also a displacement determination unit for determining the time displacement in the time/density curves and a velocity determination unit for determining the fluid velocity on the basis of the time displacement determined in the time/density curves.

At least one embodiment of an inventive computer tomography system encompasses the inventive fluid velocity determination device.

At least one embodiment of the inventive computer tomography system additionally encompasses, for example, a projection data acquisition unit. The projection data acquisition unit comprises an X-ray source and a detector system for acquiring projection measurement data from an object. Furthermore, at least one embodiment of the inventive computer tomography system also comprises a reconstruction unit for reconstructing captured projection measurement data and additionally the inventive fluid velocity determination device, wherein, in the case of at least one embodiment of the inventive computer tomography system, the reconstruction unit preferably forms part of the fluid velocity determination device.

The fundamental components of at least one embodiment of the inventive fluid velocity determination device can be realized in the majority of cases in the form of software components. This relates especially to the region definition unit, parts of the image data capture unit, the curve determination unit, the displacement determination unit, and the velocity determination unit. In principle, however, these components can also be implemented partly in the form of software-supported hardware, for example FPGAs or the like, especially if particularly fast calculations are involved. Likewise the required interfaces can be realized as software interfaces, for example if only the importing of data from other software components is involved. But they can also be realized as interfaces constructed using hardware, which are activated via suitable software.

At least one embodiment of the inventive fluid velocity determination device can especially form part of a user terminal or a control device of a CT system.

A largely software-based implementation has the advantage that previously used control devices can also be retrofitted in a simple manner via a software update in order to operate in the inventive manner. To this extent, at least one embodiment is directed to a corresponding computer program product with a computer program, which is capable of being loaded directly into a memory device of a control device of a computer tomography system, containing program sections in order to execute all the steps of at least one embodiment of the inventive method, when the program is executed in the control device. Where appropriate, a computer program product of this type can comprise, apart from the computer program, additional elements such as documentation for example and/or additional components also hardware components such as hardware keys (dongles etc.) for example, for the purpose of using the software.

For transport to the control device and/or for storage on or in the control device, use can be made of a computer-readable medium, for example a memory stick, a hard drive or some other transportable or permanently installed data medium, on which the program sections of the computer program that are capable of being read and executed by an arithmetic and logic unit of the control unit are stored. The arithmetic and logic unit can encompass one or a plurality of interoperating microprocessors or the like for this purpose, for example.

The dependent claims and also the following description respectively contain especially advantageous embodiments and developments of the invention. In this regard, especially, the claims in one claim category can also be developed analogously to the dependent claims in another claim category. Additionally, the various features of different example embodiments and claims can also be combined into new example embodiments in the context of the invention.

In one embodiment of the inventive method for determining the velocity of a fluid, the fluid comprises blood flowing through a blood vessel in the region to be investigated or the fluid comprises a contrast medium flowing through a parenchyma in the region to be investigated. The term blood vessel can be understood to mean either a section of a blood vessel, a blood vessel or a blood vessel system. Contrast media are customarily used to make fluid movements in the body of an investigation object visible. Contrast media can be administered to the object to be investigated in advance, i.e. prior to the imaging and the determination of the velocity, for example. The parenchyma involves functional tissue as opposed to the interstitial tissue, which comprises the support tissue.

In a preferred embodiment of the inventive method, a topogram of the region to be investigated is recorded in advance and the separately spaced sub regions are defined on the basis of the topogram. A topogram is a simple overview recording which reproduces the outlines and broad structures of an object to be investigated. Based on the topogram, individual image recording regions can then be defined, which are reproduced as images during the actual measurement with a CT system.

In at least one embodiment of the inventive method, the separately spaced sub regions preferably lie in various layers of the topogram as viewed in the z-direction of the imaging system, that is to say in the direction of the system axis. In this embodiment, the fluid flows in the z-direction or at least has a z-component. A straight blood vessel can be captured in a plurality of layers as an image in such a way, for example, that it lies in the z-axis of the imaging system, for example a CT system. In this embodiment, which is especially easy to implement, the path traveled by the fluid between the defined sub regions can be determined immediately from the spacing of the layers, in which the defined sub regions lie, from each other.

In an especially practical variant of at least one embodiment of the inventive method—especially if the imaging method used involves a method based on computer tomography—for the purpose of producing image data, projection measurement data is first captured over a period of time and the projection measurement data is then reconstructed into time-dependent image data.

It is especially preferred if the time-dependent intensity values comprise attenuation values. This is especially the case if the imaging method used involves a method based on computer tomography. In the case of computer tomography, X-rays emitted by an X-ray source are absorbed and attenuated by a region to be mapped, and then captured by a detector, the signal from which is correlated with the attenuation caused by the region to be mapped.

In a variant of at least one embodiment of the inventive method, which variant is especially advantageous in application, the time/density curves are determined by means of an equalization calculation based on the time-dependent intensity values. An equalization calculation of this type can be based on a parameterized model function, for example, which is adjusted to the captured intensity values using the equalization calculation. The equalization calculation can be implemented according to the least squares method, for example.

In an especially advantageous embodiment of the inventive method, the time displacement in the time/density curves can be determined on the basis of a section in a predetermined time interval of the time/density curves or the overall time/density curves. In principle, calculation of the time displacement on the basis of the overall time/density curve is the method of choice since in this case all the information relating to measurement is also included in the calculation of the time displacement. If different time/density curves diverge markedly from each other in parts, however, it can also be worthwhile restricting the process to one time section in which the divergence in the individual time/density curves, apart from the time displacement, is small.

In a special variant of at least one embodiment of the inventive method for determining the velocity of a fluid, the time displacement in the time/density curves is determined as follows: first, on the basis of an equalization calculation, a central time/density curve is determined, the assigned sub region for which lies in the center between the other sub regions. A sub region lying in the center should be understood to mean one which, relative to the other sub regions, as far as the path of the fluid through the sub regions is concerned, at least does not lie at the start or end of a chain of sub regions to be flowed through. It is especially preferred if around the same number of sub regions which the fluid flows through lie before this sub region and after this sub region.

Subsequently, a spatial displacement and a time displacement of the central time/density curve is implemented to the z-positions of the other sub regions, for example by displacement in the direction of the z-axis and also the time axis of a graph representing the attenuation values assigned to the individual sub regions as a function of the location and time. In this regard, the spatial displacement corresponds in each case simply to the displacement of the z-value of the central sub region to the z-values of the other sub regions. The displacement to the positions of the other sub regions takes place without the central time/density curve, having once been found, changing in its form, in other words this involves a pure translation. The displacement is preferably carried out in a minimizing manner in the time direction, i.e. it is carried out in the time direction so that the difference between the attenuation values assigned to the respective sub region and the displaced central time/density curve is minimal. The respective time/density curves which are assigned to the respective sub regions are defined on the basis of these displacements. For example, the time displacements can be designated as the time displacements of the maximum of the central time/density curve in the case of the translation described. If the time/density curves are available as parameterized curves, the time displacement can be read off directly on the basis of the corresponding parameters of the time/density curves.

Lastly, a central time displacement is determined on the basis of the spatial and time displacements assigned to the respective time/density curves. In this regard, an equalization calculation can preferably be carried out based on the spatial displacements and time displacements undertaken in each case. For example, given the assumption of a velocity that is constant over time, a linear relationship can be assumed between time displacements and spatial displacements. In this case, the central time displacement in the time/density curves is produced by adjusting a parameterized straight line to the time displacements and spatial displacements determined. Once again, this adjustment can be implemented by using an equalization calculation. By proceeding in this way, a plurality of sub regions can be taken into account during determination of the fluid velocity, which generally increases the accuracy of determination of the fluid velocity.

The fluid velocity can be determined especially simply by calculating the quotient of the spacing between the separately spaced sub regions and the time displacement determined in the time/density curves which are assigned to the relevant sub regions. If, for example, only two separately spaced sub regions have been defined, then the fluid velocity is produced by dividing the spacing between the two sub regions by the time displacement in the two time/density curves assigned to the sub regions. In this connection, the spacing should be considered to be the path traveled by the fluid under consideration between the two relevant sub regions. If a vessel through which a fluid is flowing, the velocity of which fluid is to be calculated, has a straight orientation, then this definition corresponds to the Euclidian spacing. If a vessel with a crooked orientation is present, however, then the spacing corresponds to the corresponding line integral along the central line of the vessel.

The time-dependent image data for the plurality of separately spaced sub regions can be produced in the context of a bolus-tracking method for example. A method of this type is usually employed to determine the starting time point for a contrast medium-supported imaging procedure. A bolus-tracking method of this type normally comprises monitoring a region expected to be flowed through by a contrast medium, by using medical imaging, and determining the time point at which the contrast medium moves through this region. If a plurality of regions are then monitored during the bolus tracking in place of only one region, then the velocity of the contrast medium can be determined on the basis of the data captured during the measurement. It is therefore possible to determine in advance, for example, a time point at which the contrast medium will arrive in an investigation region that is located at a distance from the monitored regions, and thus the starting time point for an imaging procedure can be determined and calculated very precisely in advance.

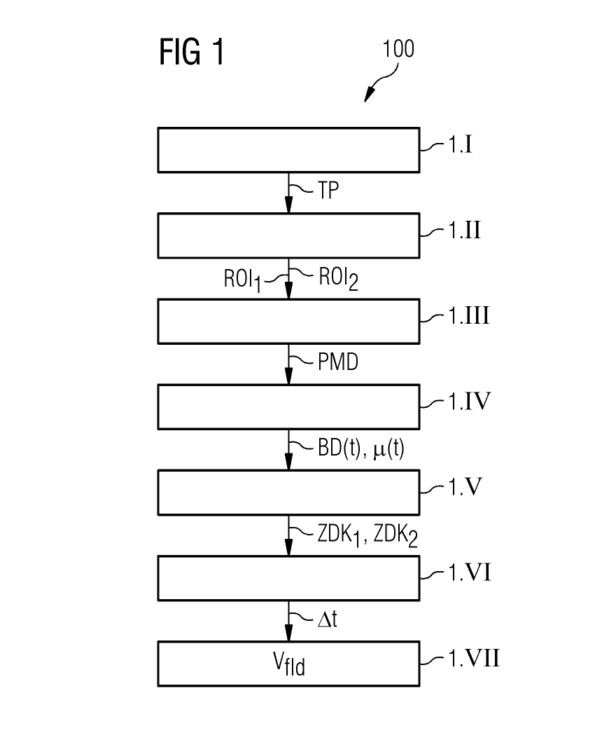

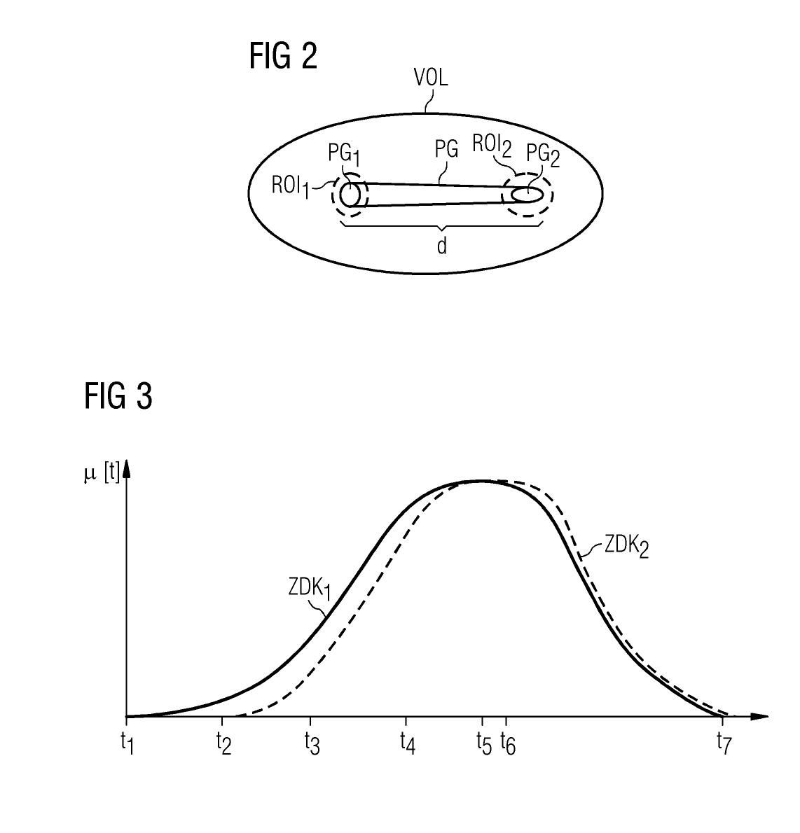

FIG. 1 shows a flow diagram illustrating a method 100 for determining a fluid velocity according to an example embodiment of the invention. In step 1.I, a topogram TP of a region VOL to be investigated, of a patient, is firstly recorded, for example with the aid of a CT system. Then, in step 1.II, sub regions ROI₁, ROI₂ to be mapped in the topogram TP (see FIG. 2), through which sub regions the fluid that is to have its velocity determined is flowing, are defined.

In step 1.III, a CT image recording is carried out, projection measurement data PMD from the sub regions ROI₁, ROI₂ to be mapped being captured over a measurement period. In step 1.IV, time-dependent image data BD(t) is reconstructed from the projection measurement data PMD. The reconstruction can be carried out by using a reconstruction method which is based on filtered back projection, for example.

In step 1.V, time/density curves ZDK₁, ZDK₂ are determined on the basis of the reconstructed time-dependent image data BD(t), or the attenuation values μ(t) comprised by the said data. Determination of the time/density curves on the basis of the attenuation values μ(t) can be implemented by using, in each case, a separate “fit” for the attenuation values μ(t) for each of the sub regions ROI₁, ROI₂ to be mapped, for example. In this context, a “fit” is intended to designate a determination of a time/density curve ZDK₁, ZDK₂ by using an equalization calculation which is applied to the measured attenuation values μ(t). For example, a family of curves can be specified for this “fit”, i.e. a parameterized function for each respective, or jointly for both, time/density curve(s). The parameters of the function for the respective time/density curve ZDK₁, ZDK₂ are identified in this context such that the overall divergence, for example the sum of the squares of the spacings of the attenuation values μ(t) for the curve to be fitted, which corresponds to the respective parameterized function, is minimal wherever possible. A parameterized function for a time/density curve can be established by means of theoretical considerations and/or on the basis of experimental data, for example.

In step 1.VI, time displacements Δt between the time/density curves ZDK₁, ZDK₂ are determined on the basis of the time/density curves ZDK₁, ZDK₂ determined. Then, in step 1.VII, the fluid velocity vfld is calculated on the basis of the following formula:

$${v}_{fld} = \frac{d}{Δ t} \qquad ( 1 )$$

where d is the spacing between the two different sub regions ROI₁, ROI₂. As already described, the spacing in the sense of the definition used here corresponds to the length of the fluid path between the two sub regions defined in step 1.II. A comprehensive description has been given as to how the blood flow velocity or the fluid velocity v_{fld} in general can be determined. Other variables can, in turn, also be derived (indirectly) from this, however, such as the pressure, for example, prevailing in an investigated blood vessel, for example.

FIG. 2 shows a region VOL to be investigated, of an investigation object, from the perspective of the z-direction. Furthermore, the ends PG1, PG2 of a blood vessel PG running in the horizontal direction can be recognized, which ends form part of a layer to be mapped which lies at right angles to the z-direction, i.e. in the plane of the paper. The two ends PG1, PG2 are distanced from each other with a spacing d to be measured. As already mentioned a number of times, the spacing is to be understood as the flow path of a fluid between the two ends PG1, PG2. Two sub regions ROI₁, ROI₂ to be mapped can also be recognized in FIG. 2, comprising the two ends PG1, PG2 of the blood vessel PG through which the blood with the velocity to be determined is flowing. As described in connection with the method 100 illustrated in FIG. 1, these two sub regions ROI₁, ROI₂ to be mapped are defined on the basis of the recording of a topogram prior to projection measurement data (PMD) being captured from these sub regions ZDK₁, ZDK₂, from which PMD image data is reconstructed during the method 100, on the basis of which image data the blood flow velocity vfld is in turn determined during the method 100.

FIG. 3 shows the time profile of, for example, attenuation values μ(t) corresponding to a contrast medium concentration at two different points of a vascular system following the injection of a contrast medium into the vascular system, i.e. for two different first and second sub regions ROI₁, ROI₂ which are arranged at different vessel sections (see FIG. 2) of the vascular system of a patient, for example. The graph has been prepared on the basis of attenuation values μ(t) measured in the blood vessel PG (see FIG. 2) by using a CT system, the attenuation values μ(t) representing attenuation values μ(t) averaged over the respective sub region ROI₁, ROI₂, for example. In FIG. 3, the time profile of the attenuation values μ(t) in the two sub regions ROI₁, ROI₂ is illustrated graphically by means of time/density curves ZDK₁, ZDK₂. In more accurate terms, the time/density curves ZDK₁, ZDK₂ shown are curves fitted to the captured image data or attenuation values by means of an equalization calculation.

The time profile of the time/density curves ZDK₁, ZDK₂ shown in FIG. 3 can be interpreted as follows: the heart pumps the blood through the vascular system with a constant cardiac output per unit of time at an average velocity v_{fld}. Following the injection of a contrast medium at a first time point t₁, the contrast medium concentration in the first end PG₁ of the blood vessel of the system in the first sub region ROI₁ (see FIG. 2) firstly increases, for example. This change corresponds to the rise in a first time/density curve ZDK₁ in FIG. 3, which is illustrated with a solid line. Later, the contrast medium concentration in the first end PG1 of the blood vessel decreases again. After a certain time lag t₂-t₁, the contrast medium concentration also increases at the second end PG₂ of the blood vessel PG in the system, at the position of the second sub region ROI₂, with effect from a second time point t₂. This behavior is represented in FIG. 3 by means of a second time/density curve ZDK₂ which is shown as an intermittent line. With effect from a third time point t₃, the two time/density curves ZDK₁, ZDK₂ run approximately parallel up to a fourth time point t₄. In this special case, this region is most suitable for a determination of a time displacement of the time/density curves ZDK₁, ZDK₂. From a fifth time point t₅ onward, the first time/density curve ZDK₁ falls, i.e. the corresponding attenuation values μ(t) decrease with time t. At a sixth time point t₆, the two time/density curves ZDK₁, ZDK₂ intersect and then both fall to a seventh time point t₇, at which the CT image recording was finished. For the time interval between the third time point t₃ and the fourth time point t₄ especially, a time displacement Δt between the two curves can be determined quite well.

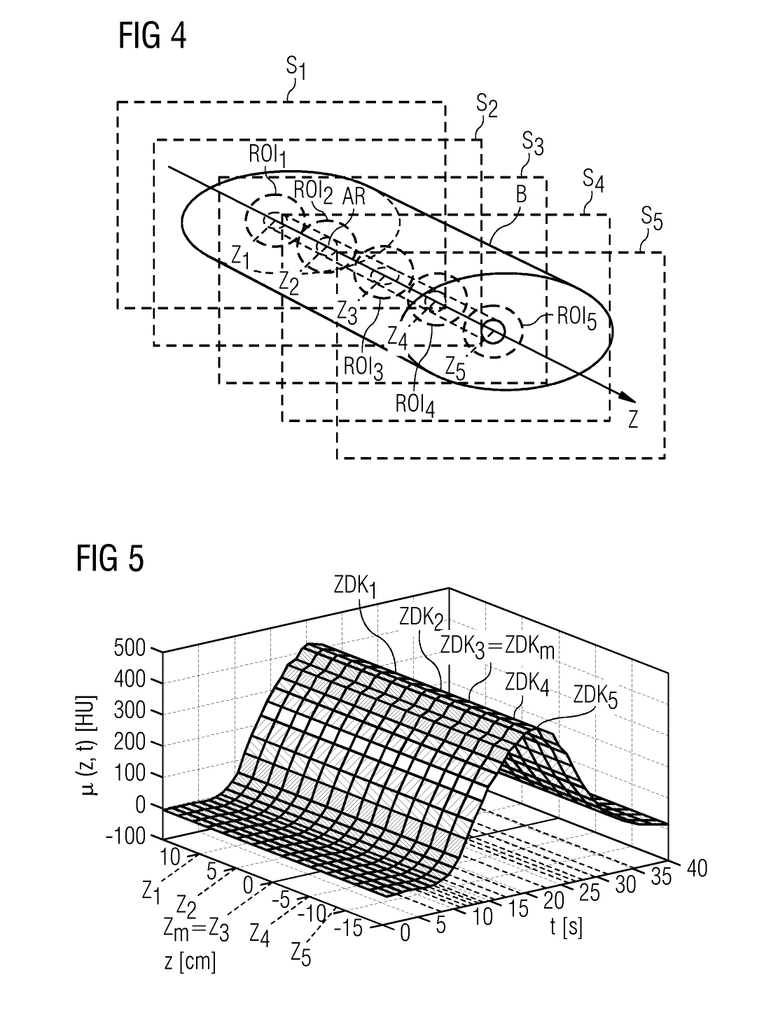

FIGS. 4 to 6 illustrate a fluid velocity determination according to a second example embodiment. In structural terms, the approach corresponds to the method 100, the approach being somewhat different in detail during the determination of the time/density curves and also the determination of the time displacement, however.

FIG. 4 shows a region VOL to be investigated, in an investigation object, in this case a leg, in a perspective view. A section of leg B with an artery AR or a section of this artery AR can be recognized. Purely for the purpose of simplicity, the artery AR runs on the z-axis in the z-direction, i.e. in the direction of the system axis. Five layers S₁ . . . S₅ are also shown by means of intermittent lines at five different z-positions z₁ . . . z₅, in which layers five regions ROI₁ . . . ROI₅ to be mapped are defined on the basis of a topogram for example, through which regions the artery AR runs in each case. The five defined layers S₁ . . . S₅ or the sub regions arranged inside them are captured in image form during the following imaging.

FIG. 5 illustrates the attenuation values μ(z,t) from a CT recording for the five regions ROI₁ . . . ROI₅ to be mapped as shown in FIG. 4 at the five different z positions z₁ . . . z₅ (and many further z positions) by means of time/density curves ZDK₁ . . . ZDK₅. These time/density curves are slightly displaced in the time direction. The time/density curves are created as follows for example: a central time/density curve ZDK₃ is first determined for the third z-position z₃ by using an equalization calculation, i.e. a parameterized model curve is fitted to the measured attenuation values μ(z,t). Then this central time/density curve ZDK₃ is displaced in the z-direction to each of the other positions z₁, z₂, z₄, z₅ and additionally displaced in the time direction such that the central time/density curve ZDK₃=ZDKₘ displays an optimal fit to the attenuation values present at the respective positions. The optimal time position in each case be effected by using a simple numerical minimization or a corresponding equalization calculation. The central time/density curve ZDKₘ displaced in this way ultimately forms the respective other time/density curves ZDK₁, ZDK₂, ZDK₄, ZDK₅. Except for the different time positions and z-positions, therefore, the five time/density curves ZDK₁, ZDK₂, ZDK₄, ZDK₅ in this embodiment are realized in the same way.

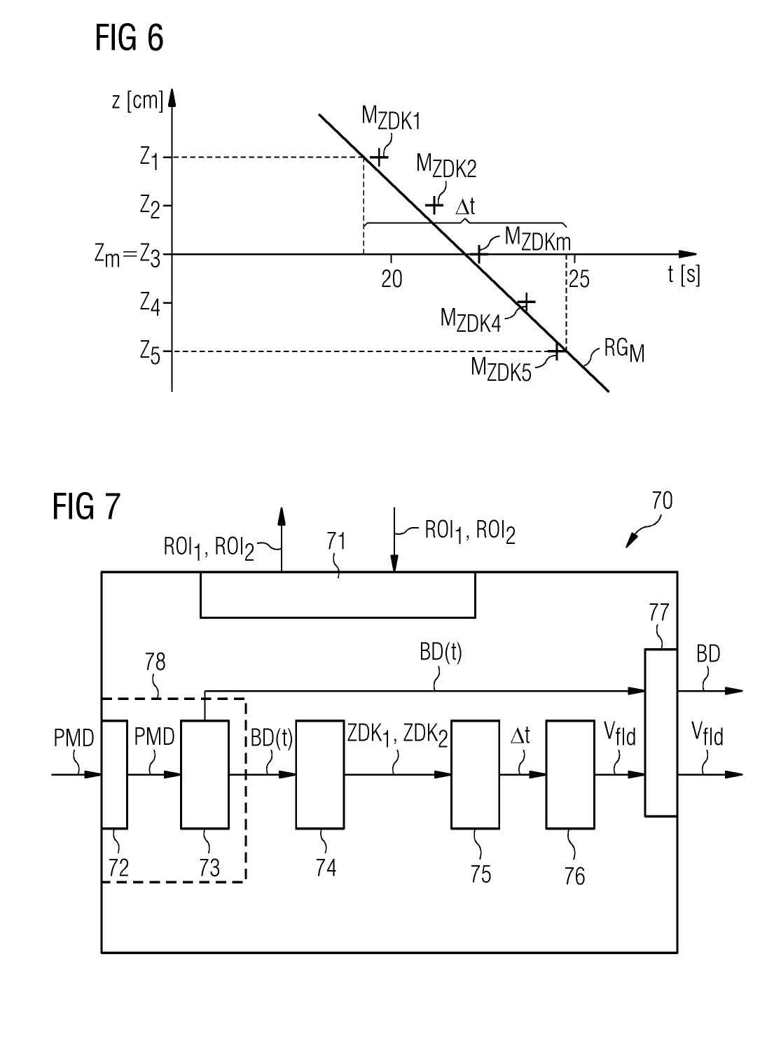

The time displacement of the five time/density curves ZDK₁ . . . ZDK₅ illustrated in FIG. 5 is illustrated in FIG. 6, which shows the graph in FIG. 5 from above, i.e. as viewed from the direction of the axis representing the attenuation values μ(z,t). With regard to the individual time/density curves, their maxima M_{ZDK1} . . . M_{ZDK2} are marked in the graph in FIG. 6. These maxima are offset in the time direction and are approximated by means of a best-fit line RG_{M}, which can be determined on the basis of the captured data by using an equalization calculation. A time displacement Δt=t₁−t₅ corresponds to the spacing Δz=z₅−z₁ between the first time/density curve ZDK₁ and the fifth time/density curve ZDK₅, which displacement can be read off from the best-fit line RG_{M}. Lastly, the fluid velocity v_{fld} is produced from the quotient of the two displacement values Δz, Δt to give:

$${v}_{fluid} = \frac{Δ z}{Δ t} . \qquad ( 2 )$$

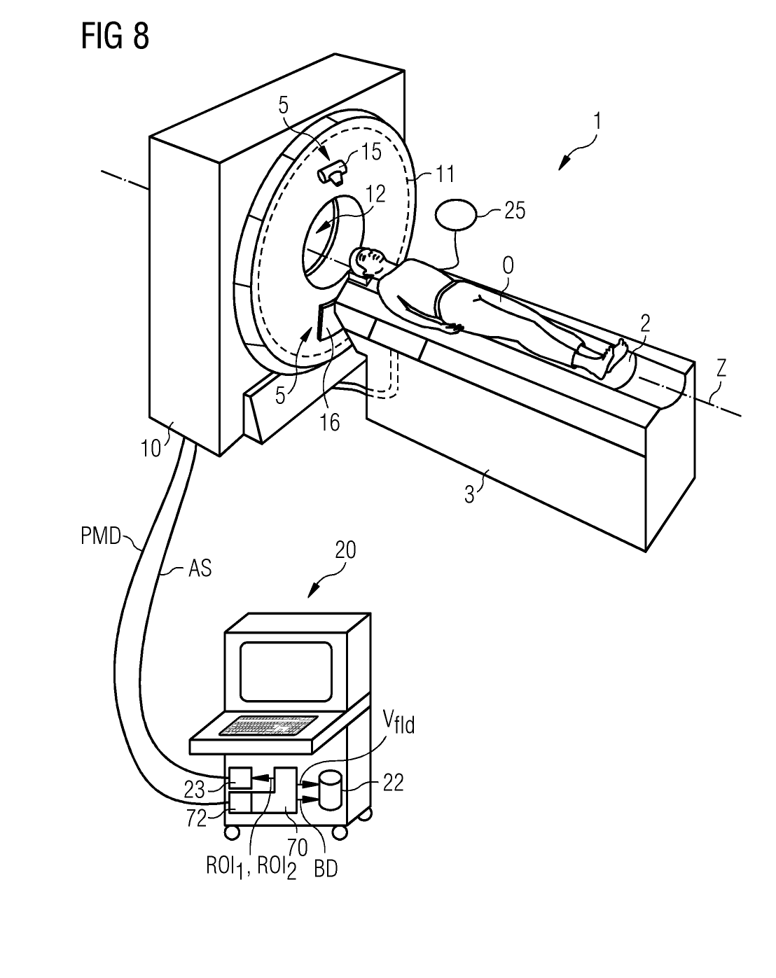

FIG. 7 shows a fluid velocity determination device 70. The fluid velocity determination device 70 can form part of a control device of a CT system 1, for example, as shown in FIG. 8. The fluid velocity determination device 70 comprises a region definition unit 71 for defining a plurality of separately spaced sub regions ROI₁, ROI₂ through which the fluid with the velocity v_{fld} to be determined is flowing. The region definition unit 71 obtains information about the definition or position of the sub regions ROI₁, ROI₂, for example from an input by a user or also in an automated manner, and transfers this information in a form to be processed by an activation unit 23 (see FIG. 8). The activation unit 23 then controls a measurement device of a CT system (see FIG. 8) on the basis of the information obtained so that the predetermined sub regions ROI₁, ROI₂ are mapped or projection measurement data for sub regions is recorded.

Apart from this, the fluid velocity determination device 70 comprises an image data capture unit 78 which has a projection measurement data capture unit 72 in this embodiment, which unit captures projection measurement data PMD generated during an imaging procedure. Furthermore, the image data capture unit 78 comprises a reconstruction unit 73 which is set up to reconstruct time-dependent image data BD(t) for the plurality of separately spaced sub regions ROI₁, ROI₂ on the basis of the captured projection measurement data PMD. The image data BD(t) determined is transferred to an output interface 77 from where it is forwarded to connected units such as a memory unit or a terminal for example. Additionally, the reconstructed image data BD(t) is also transferred to a curve determination unit 74, which determines time/density curves ZDK₁, ZDK₂ corresponding to a plurality of time-dependent intensity values μ(t) on the basis of the time-dependent image data BD(t) for the separately spaced sub regions ROI₁, ROI₂. Then the data referring to the time/density curves ZDK₁, ZDK₂ is transferred to a displacement determination unit 75, which determines from it a time displacement Δt in the time/density curves ZDK₁, ZDK₂. The data referring to the time displacement Δt determined is subsequently forwarded to a velocity determination unit 76, which determines a fluid velocity v_{fld} based on the time displacement Δt determined in the time/density curves ZDK₁, ZDK₂. Lastly the value for the fluid velocity v_{fld} is transferred to the output interface 77 mentioned previously, from where this information is forwarded to connected units such as a memory unit or a terminal for example (see FIG. 8).

FIG. 8 shows a computer tomography system 1 which comprises the fluid velocity determination device 70 shown in FIG. 7. In this regard, the CT system 1 consists essentially of a customary scanner 10, in which, on a gantry 11, a projection data acquisition unit 5 containing a detector 16 and an X-ray source 15 located opposite the detector 16 rotates around a measurement space 12. Situated in front of the scanner 10 is a patient support device 3 or a patient table 3, the upper part 2 of which, with a patient O situated on it, can be displaced to the scanner 10 in order to move the patient O through the measurement space 12 relative to the detector system 16. The scanner 10 and the patient table 3 are activated by means of a control device 20, from where acquisition control signals AS come by way of a customary activation unit 23 containing a control interface, in order to activate the overall system according to specified measurement protocols in the conventional manner. With regard to image recording in the context of the inventive method 100, data referring to sub regions ROI₁, ROI₂ to be mapped is also transferred to the activation unit 23 either directly by means of input by a user or indirectly by way of the inventive fluid velocity determination device 70 (see also FIG. 7). In the event of spiral acquisition, the movement of the patient O along the z-direction, which corresponds to the system axis z running longitudinally through the measurement space 12, and the simultaneous rotation of the X-ray source 15 creates a helical path for the X-ray source 15 relative to the patient O during the measurement procedure. In parallel, the detector 16 is always also present opposite the X-ray source 15 in this regard, in order to capture projection measurement data PMD which is then used for reconstructing volume and/or layer image data. A sequential measurement method is likewise also possible, in which a fixed position in the z-direction is traversed to and then in the course of a rotation, a partial rotation or a plurality of rotations at the relevant z-position, the required projection measurement data PMD is captured in order to reconstruct a sectional image at this z-position or to reconstruct image data from the projection data of a plurality of z-positions. In principle, the inventive method can also be employed on other CT systems, e.g. with a plurality of X-ray sources and/or detectors and/or with a detector forming a complete ring. For example, the inventive method can also be applied on a system with a non-moving patient table and a gantry moved in the z-direction (a so-called sliding gantry).

The projection measurement data PMD (also referred to in the following as raw data) acquired by the detector 16 is passed on to the control device 20 by way of a raw data interface 72, which in this embodiment forms part of the fluid velocity determination device 70. Following suitable pre-processing (e.g. filtering and/or beam hardening correction) where appropriate, this raw data is then subjected to further processing in the fluid velocity determination device 70 according to an example embodiment of the invention in the manner described above. In this example embodiment, the fluid velocity determination device 70 is implemented in the control device 20, largely in the form of software (except for the interfaces to the units connected to it), on a processor.

The data referring to the fluid velocity v_{fld} determined by the fluid velocity determination device 70 and also the captured image data BD is deposited in a memory 22 of the control device 20 and/or output on the screen of the control device 20 in the usual way. However, the data can also be fed, by way of an interface not shown in FIG. 8, into a network connected to the computer tomography system 1, for example a Radiological Information System (RIS), and deposited in a mass storage device accessible there or output to printers or filming stations connected there. The data can thus be subjected to further processing as required and then stored or output.

Additionally, a contrast medium injection device 25 is also shown in the drawing in FIG. 8, with the aid of which device the patient P is injected with a contrast medium in advance, i.e. prior to the inventive method 100, the behavior of which medium is captured in image form by using the computer tomography system 1.

The components of the fluid velocity determination device 70 can be implemented in the majority of cases, or entirely, in the form of software elements on a suitable processor. The interfaces between these components especially can also be realized purely in terms of software. All that is required is the existence of access capabilities to suitable memory regions in which the data can be suitably put into intermediate storage and called up again and updated at any time.

In conclusion, it is pointed out once again that the methods and devices described above merely constitute preferred example embodiments of the invention and that the invention can be varied by a person skilled in the art without departing from the scope of the invention to the extent that it is specified by the claims. The method and the fluid velocity determination device have thus been primarily explained on the basis of a computer tomography system for recording medical image data. However, the invention is not restricted to application in computer tomography nor to application in the medical domain; instead, the invention can also be applied in principle to other imaging systems, such as magnetic resonance tomography systems for example, and also to the recording of images for other purposes. For the sake of completeness, it is also pointed out that the use of the indefinite article “a” or “an” does not exclude the possibility of the relevant features also being present in multiple form. Likewise the term “unit” does not exclude the possibility of the said unit consisting of a plurality of components, which can also be spatially distributed where appropriate.

The aforementioned description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure and appended claims.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one embodiment of the invention relates to a non-transitory computer-readable storage medium comprising electronically readable control information stored thereon, configured in such that when the storage medium is used in a controller of a magnetic resonance device, at least one embodiment of the method is carried out.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining velocity of a fluid in a region to be investigated using an imaging method of an investigation object, comprising:
defining a plurality of separately spaced sub regions of a region to be investigated, through which sub regions the fluid is flowing;
producing time-dependent image data for the plurality of separately spaced sub regions;
respectively determining time/density curves with a plurality of time-dependent intensity values on the basis of the time-dependent image data for each of the separately spaced sub regions;
determining a time displacement in the time/density curves; and
determining the fluid velocity on the basis of the time displacement determined in the time/density curves.

2. The method of claim 1, wherein the fluid comprises at least one of blood and a contrast medium flowing through a blood vessel in the region to be investigated or the fluid comprises a contrast medium flowing through a parenchyma in the region to be investigated.

3. The method of claim 1, wherein a topogram of the region to be investigated is recorded in advance and the separately spaced sub regions are defined on the basis of the topogram.

4. The method of claim 1, wherein the separately spaced sub regions lie in various layers of the topogram as viewed in the z-direction of the imaging system.

5. The method of claim 1, wherein for the purpose of producing image data, projection measurement data is first captured over a period of time and the projection measurement data is then reconstructed into time-dependent image data.

6. The method of claim 1, wherein the time-dependent intensity values comprise attenuation values.

7. The method of claim 1, wherein the time/density curves are determined by use of an equalization calculation based on the time-dependent intensity values.

8. The method of claim 1, wherein the time displacement in the time/density curves is determined on the basis of a section of the time/density curves in a time interval or on the basis of the overall time/density curves.

9. The method of claim 1, wherein the time displacement in the time/density curves is determined with the aid of at least the following:
determining a central time/density curve, the assigned sub region for which lies in the center between the other sub regions, on the basis of an equalization calculation,
implementing a spatial displacement and a time displacement of the central time/density curve to the positions of the other sub regions so that the difference between the intensity values assigned to the respective sub region and the displaced central time/density curve is minimal,
defining a respective time/density curve for each of the other sub regions based on the respective spatial and time displacement undertaken, and
determination of the time displacement as the central time displacement on the basis of the spatial and time displacements assigned to the respective time/density curves.

10. The method of claim 9, wherein for the purpose of determining the central time displacement, an equalization calculation is carried out on the basis of the spatial and time displacements assigned to the respective time/density curves.

11. The method of claim 1, wherein the fluid velocity is determined by calculating the quotient of the spacing between the separately spaced sub regions and the time displacement determined in the time/density curves.

12. The method of claim 1, wherein the time-dependent image data for the plurality of separately spaced sub regions is produced in the context of a bolus-tracking method.

13. A fluid velocity determination device, comprising:
a region definition unit (71) for defining a plurality of separately spaced sub regions (ROI₁, ROI₂) of a region (VOL) to be investigated, through which sub regions the fluid is flowing;
an image data capture unit to produce time-dependent image data for the plurality of separately spaced sub regions;
a curve determination unit to determine time/density curves for, in each case, a plurality of time-dependent intensity values on the basis of the time-dependent image data for the respective separately spaced sub regions (ROI₁, ROI₂);
a displacement determination unit to determine the time displacement in the time/density curves; and
a velocity determination unit to determine the fluid velocity on the basis of the time displacement determined in the time/density curves.

14. A computer tomography system, comprising:
the fluid velocity determination device of claim 13.

15. A non-transitory computer program product including a computer program, loadable directly into a memory device of a control device of a computer tomography system, including program sections to execute the method of claim 1 when the computer program is executed in the control device of the computer tomography system.

16. A non-transitory computer-readable medium including program sections that are readable and executable by an arithmetic and logic unit, stored in order to execute the method of claim 1 when the program sections are executed by the arithmetic and logic unit.

17. The method of claim 2, wherein a topogram of the region to be investigated is recorded in advance and the separately spaced sub regions are defined on the basis of the topogram.

18. The method of claim 2, wherein the time displacement in the time/density curves is determined with the aid of at least the following:
determining a central time/density curve, the assigned sub region for which lies in the center between the other sub regions, on the basis of an equalization calculation,
implementing a spatial displacement and a time displacement of the central time/density curve to the positions of the other sub regions so that the difference between the intensity values assigned to the respective sub region and the displaced central time/density curve is minimal,
defining a respective time/density curve for each of the other sub regions based on the respective spatial and time displacement undertaken, and
determination of the time displacement as the central time displacement on the basis of the spatial and time displacements assigned to the respective time/density curves.

19. The method of claim 18, wherein for the purpose of determining the central time displacement, an equalization calculation is carried out on the basis of the spatial and time displacements assigned to the respective time/density curves.

20. The method of claim 4, wherein the time displacement in the time/density curves is determined with the aid of at least the following:
determining a central time/density curve, the assigned sub region for which lies in the center between the other sub regions, on the basis of an equalization calculation,
implementing a spatial displacement and a time displacement of the central time/density curve to the positions of the other sub regions so that the difference between the intensity values assigned to the respective sub region and the displaced central time/density curve is minimal,
defining a respective time/density curve for each of the other sub regions based on the respective spatial and time displacement undertaken, and
determination of the time displacement as the central time displacement on the basis of the spatial and time displacements assigned to the respective time/density curves.

21. The method of claim 20, wherein for the purpose of determining the central time displacement, an equalization calculation is carried out on the basis of the spatial and time displacements assigned to the respective time/density curves.

22. A non-transitory computer-readable medium including program sections that are readable and executable by an arithmetic and logic unit, stored in order to execute the method of claim 2 when the program sections are executed by the arithmetic and logic unit.

23. A non-transitory computer-readable medium including program sections that are readable and executable by an arithmetic and logic unit, stored in order to execute the method of claim 4 when the program sections are executed by the arithmetic and logic unit.
